(19) United States
(12) Patent Application Publication
(10) Pub. No.: US 2024/0407835 A1
(43) Pub. Date: Dec. 12, 2024

(54) LAYERED CORE INSULATED GUIDEWIRE

(71) Applicant: Boston Scientific Scimed, Inc., Maple Grove, MN (US)

(72) Inventors: Edward Louis Gonda, Minneapolis, MN (US); Brice Lee Shireman, Maple Grove, MN (US); Spencer Fodness-Bondhus, Columbia Heights, MN (US)

(21) Appl. No.: 18/680,420

(22) Filed: May 31, 2024

Related U.S. Application Data
(60) Provisional application No. 63/507,169, filed on Jun. 9, 2023.

(51) Int. Cl. A61B 18/14; A61B 17/00; A61B 18/00; A61B 90/00; A61M 25/09

(52) U.S. Cl. CPC A61B 18/1492; A61B 2017/00247; A61B 2018/00083; A61B 2090/3966; A61M 2025/09083; A61M 2025/09166; A61M 2025/09183

(57) ABSTRACT

A transseptal access system is disclosed. The system includes a radiofrequency perforation device including a core, a functional tip coupled to a distal end of the core, and a conductor extending along a length of the core. The conductor is insulated along a portion of its length and is electrically coupled to the functional tip.

### CROSS REFERENCE TO RELATED APPLICATIONS

The present application claims priority to U.S. Provisional Application No. 63/507, 169 entitled “LAYERED CORE INSULATED GUIDEWIRE,” filed Jun. 9, 2023, the disclosure of which is incorporated herewith by reference in its entirety.

### TECHNICAL FIELD

The present invention relates generally to methods and devices usable to deliver energy within the body of a patient. More specifically, the present invention is concerned with a radiofrequency perforation apparatus.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongate needle, which is structured such that it may be introduced into a body of the patient via the femoral vein and directed towards the heart. This device relies on the use of mechanical force to drive the sharp tip through the septum.

Alternatively, radiofrequency perforation apparatuses have been developed, whereby the septal perforation is accomplished by the application of focused radiofrequency energy to the septal tissue via an electrode at the distal end of a relatively thin conductive probe.

Against this background, there exists a continuing need in the industry to provide improved radiofrequency perforation devices and methods. An object of the present invention is therefore to provide such a radiofrequency perforation apparatus.

### SUMMARY

Example 1 is a transseptal access system comprising a radiofrequency perforation device including a core, a functional tip coupled to a distal end of the core, and a conductor extending along a length of the core; wherein the conductor is insulated along a portion of its length and is electrically coupled to the functional tip.

Example 2 is the transseptal access system of Example 1 the core further comprising a core lumen.

Example 3 is the transseptal access system of Example 1 the radiofrequency perforation device further comprising an outer coil wound around a distal portion of the core.

Example 4 is the transseptal access system of Example 3 further comprising a second electrode, wherein the outer coil is electrically coupled to the second electrode.

Example 5 is the transseptal access system of Example 1 wherein the core is a hypotube.

Example 6 is the transseptal access system of Example 1 wherein the core is made from a non-conductive material.

Example 7 is the transseptal access system of Example 1 wherein the conductor is made from a conductive material and at least one of an outer surface of the core and an inner surface of the core is at least partially covered by an insulating material.

Example 8 is the transseptal access system of Example 3 wherein the outer coil is positioned on the distal most portion of the radiofrequency perforation device proximal to the functional tip.

Example 9 is the transseptal access system of Example 3 wherein the outer coil includes an outer insulative layer.

Example 10 is the transseptal access system of Example 3 wherein the outer coil includes an inner conductive layer.

Example 11 is the transseptal access system of Example 1 wherein the core includes a helical groove extending along an outer surface and further wherein the conductor is disposed at least partially in and along the helical groove.

Example 12 is the transseptal access system of Example 3 wherein the outer coil is made from a radiopaque metal material covered by an insulating layer.

Example 13 is the transseptal access system of Example 3 wherein the outer coil is made from a polymeric material doped with a radiopaque material.

Example 14 is the transseptal access system of Example 1 wherein the core is selected from the group consisting of polytetrafluoroethylene (PTFE), fluorinated ethylene propylene (FEP), polyetheretherketone (PEEK), polyethylene terephthalate (PET), and polyimide.

Example 15 is the transseptal access system of Example 1 wherein the conductor is selected from the group consisting of copper, platinum iridium, copper cladded stainless steel, platinum core stainless steel, tungsten copper alloy, and molybdenum copper alloy.

Example 16 is a transseptal access system comprising a radiofrequency perforation device including a core, a functional tip coupled to a distal end of the core, and a conductor extending along a length of the core; wherein the conductor is insulated along a portion of its length and is electrically coupled to the functional tip.

Example 17 is the transseptal access system of Example 16 the core further comprising a core lumen.

Example 18 is the transseptal access system of Example 16 the radiofrequency perforation device further comprising an outer coil wound around a distal portion of the core.

Example 19 is the transseptal access system of Example 18 further comprising a second electrode, wherein the outer coil is electrically coupled to the second electrode.

Example 20 is the transseptal access system of Example 16 wherein the core is a hypotube.

Example 21 is the transseptal access system of Example 16 wherein the core is made from a non-conductive material.

Example 22 is the transseptal access system of Example 16 wherein the conductor is made from a conductive material and at least one of an outer surface of the core and an inner surface of the core is at least partially covered by an insulating material.

Example 23 is the transseptal access system of Example 18 wherein the outer coil is positioned on the distal most portion of the radiofrequency perforation device proximal to the functional tip.

Example 24 is the transseptal access system of Example 16 wherein the core includes a helical groove extending along an outer surface and further wherein the conductor is disposed at least partially in and along the helical groove.

Example 25 is the transseptal access system of Example 18 wherein the outer coil is made from a radiopaque metal material covered by an insulating layer.

Example 26 is the transseptal access system of Example 18 wherein the outer coil is made from a polymeric material doped with a radiopaque material.

Example 27 is the transseptal access system of Example 16 wherein the core is selected from the group consisting of polytetrafluoroethylene (PTFE), fluorinated ethylene propylene (FEP), polyetheretherketone (PEEK), polyethylene terephthalate (PET), and polyimide.

Example 28 is the transseptal access system of Example 16 wherein the conductor is selected from the group consisting of copper, platinum iridium, copper cladded stainless steel, platinum core stainless steel, tungsten copper alloy, and molybdenum copper alloy.

Example 29 is a transseptal access system comprising a radiofrequency perforation device including a core having a core lumen, a functional tip coupled to a distal end of the core, a conductor extending along a length of the core, and an outer coil wound around a distal portion of the core; wherein the conductor is insulated along a portion of its length and is electrically coupled to the functional tip.

Example 30 is the transseptal access system of Example 29 further comprising a second electrode, wherein the outer coil is electrically coupled to the second electrode.

Example 31 is the transseptal access system of Example 29 wherein the core is a hypotube, and wherein the core is made from a non-conductive material.

Example 32 is the transseptal access system of Example 29 wherein the conductor is made from a conductive material and at least one of an outer surface of the core and an inner surface of the core is at least partially covered by an insulating material.

Example 33 is the transseptal access system of Example 29 wherein the outer coil is positioned on the distal most portion of the radiofrequency perforation device proximal to the functional tip.

Example 34 is the transseptal access system of Example 29 wherein the core includes a helical groove extending along an outer surface and further wherein the conductor is disposed at least partially in and along the helical groove.

Example 35 is the transseptal access system of Example 29 wherein the outer coil is made from a radiopaque metal material covered by an insulating layer, and wherein the outer coil is made from a polymeric material doped with a radiopaque material.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the present disclosure.

FIG. 2 is a schematic illustration of a dilator and radiofrequency perforation device of the transseptal access system illustrated in FIGS. 1A-1C, according to embodiments of the present disclosure.

FIGS. 3A-3C are schematic illustrations of a distal end portion of a radiofrequency perforation device with a multi-layered insulated core providing dielectric potential to an energized guidewire of the transseptal access system illustrated in FIGS. 1A-1C, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

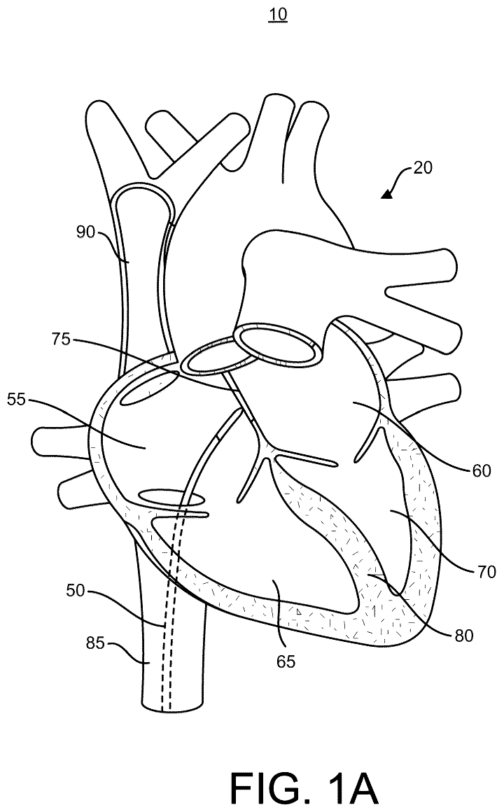

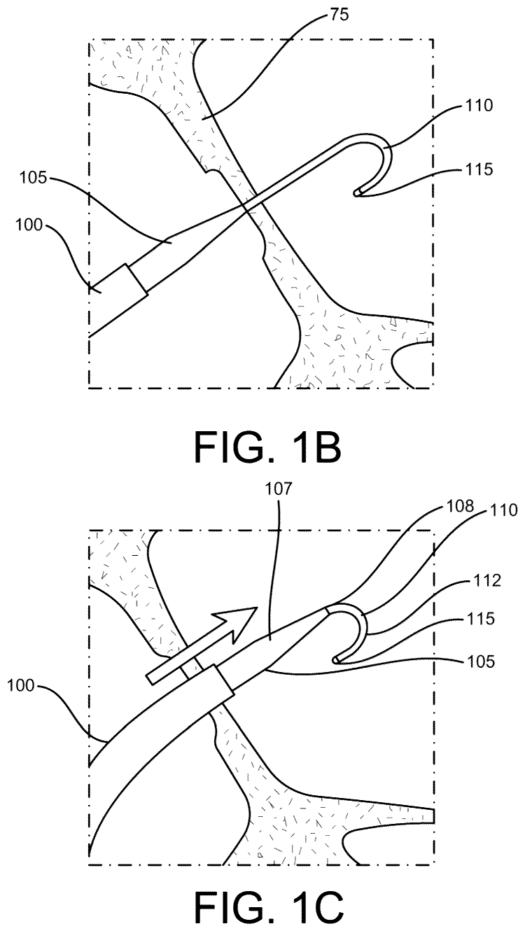

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75, and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A and 1B is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110 having distal end portion 112 terminating in a tip electrode 115. As shown, in the assembled use state illustrated in FIGS. 1A and 1B, the RF perforation device 110 can be disposed within the dilator 105, which itself can be disposed within the sheath 100. In one embodiment in which the transseptal access system 50 is deployed into the right atrium 55 via the IVC 85, a user introduces a guidewire (not shown but explained in greater detail herein) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are advanced so that their distal ends are positioned in the right atrium 55. The RF perforation device 110 described can then be introduced into the dilator 105, and advanced toward the heart 20.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape or pigtail shape. Examples of such RF perforation devices can be found, for example, in U.S. patents application Ser. No. 16/445,790 and Ser. No. 16/346,404 assigned to Baylis Medical Company, Inc. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device into the left atrium 60. The anchoring function of the pre-formed distal end portion 112 inhibits unintended retraction of the distal end portion 112, and corresponding loss of access to the perforated site on the atrial septum 75, during such withdrawal.

The present disclosure describes novel devices and methods for providing transseptal access to the left atrium 60 using radiofrequency energy. As will be explained in greater detail herein, the embodiments of the present disclosure describe a layered core insulated guidewire that may simplify the means of providing electrical connectivity between the radiofrequency puncture device and radiofrequency energy generator, while providing enhanced manipulability by the user.

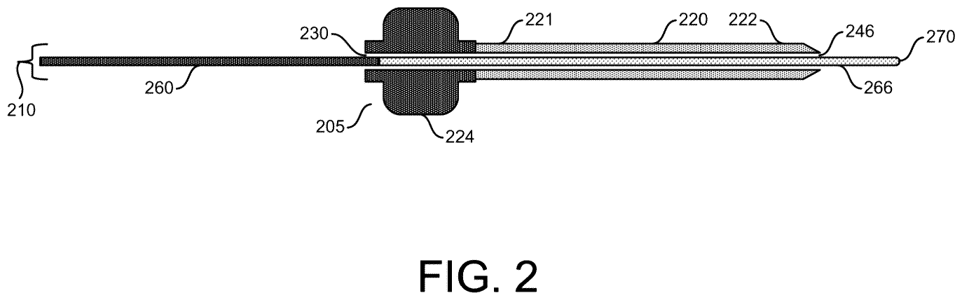

FIG. 2 is an illustration of a dilator 205 and an RF perforation device 210 according to an embodiment of the present disclosure. As shown, the dilator 205 includes a dilator body 220, a dilator hub 224, and a dilator lumen 230 extending longitudinally through the hub 224 and the dilator body 220. Additionally, the dilator body 220 has a proximal end portion 221 and an opposite distal end portion 222 terminating in a distal tip 246. The hub 224 is attached to the proximal end portion 221 of the dilator body 220. While the perforation device in FIG. 2 is described as a radiofrequency perforation device, in embodiments, the perforation device may be a mechanical perforation device.

As can be further seen from FIG. 2, the RF perforation device 210 includes a proximal portion 260 and a distal portion 266 extending from the proximal portion 260 and terminating in a distal functional tip 270 (e.g., a tip electrode such as described above in connection with FIGS. 1A-1C). As will be appreciated, the length of the RF perforation device 210 is greater than the length of the dilator 205 so that part of the proximal portion 260 of the RF perforation device 210 extends proximally of the hub 224 when the distal portion 266, particularly the functional tip 270, extends distally of the dilator 205, thus allowing the proximal portion 260 to be manipulated by the user as needed.

In embodiments, the proximal portion 260 of the RF perforation device 210 has an electrically insulated outer surface. As such, the proximal portion 260 can be handled directly by the user when the RF perforation device 210 is energized. In the illustrated embodiment, the proximal portion 260 is of a unitary construction formed entirely of an electrically insulative material. One exemplary class of materials for construction of the proximal portion can include various grades of polytetrafluoroethylene (PTFE), polyetheretherketone (PEEK), among others. In embodiments, the proximal portion 260 can further include reinforcing elements, e.g., a polymeric braid or coil, to enhance the structural properties, e.g., stiffness, torque transfer capability, and the like.

In the illustrated embodiment, the distal portion 266 is electrically conductive and is capable of transferring radiofrequency energy supplied by an external RF generator to the functional tip 270 for subsequent delivery to the target tissue in a transseptal crossing procedure, as described above. Any biocompatible electrically conductive material may be selected for construction of the distal portion 266. Exemplary materials may include stainless steel, nickel-titanium alloy, and the like. Further, for ease of illustration, the distal portion 266 is depicted in FIG. 2 as a single solid structure, although the construction of the distal portion 266 can vary to accommodate the particular structural requirements for the RF perforation device 210, as will be further explained below. For example, in embodiments, the distal portion 266 can be constructed as a solid rod, a tube or a coil.

Additionally, in embodiments, the distal portion 266 can be constructed in multiple segments, e.g., a solid rod or hypotube in the regions nearest the proximal portion 260, and a coiled structure more distally to provide enhanced flexibility and torqueability. In embodiments, the distal portion can have a composite construction, e.g., a solid or tubular core conductor surrounded by a wire coil. Additionally in the illustrated embodiment, the proximal and distal portions 260, 266 are substantially isodiametric, although this is not a strict requirement in all embodiments.

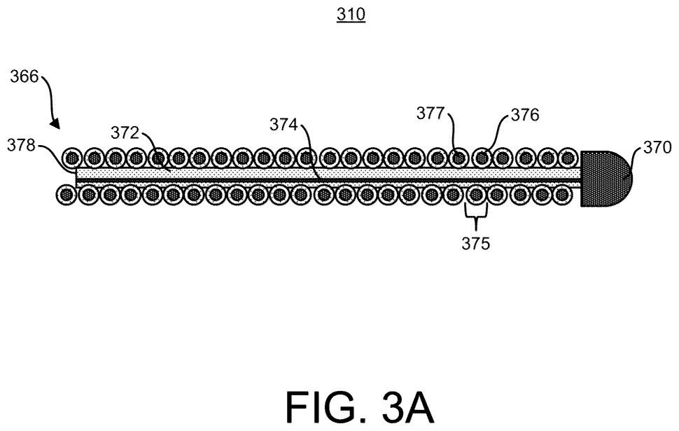

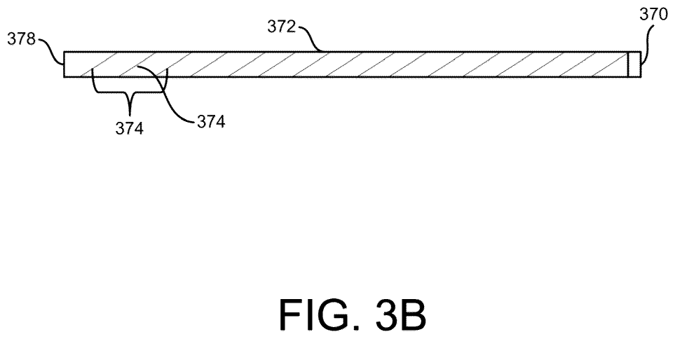

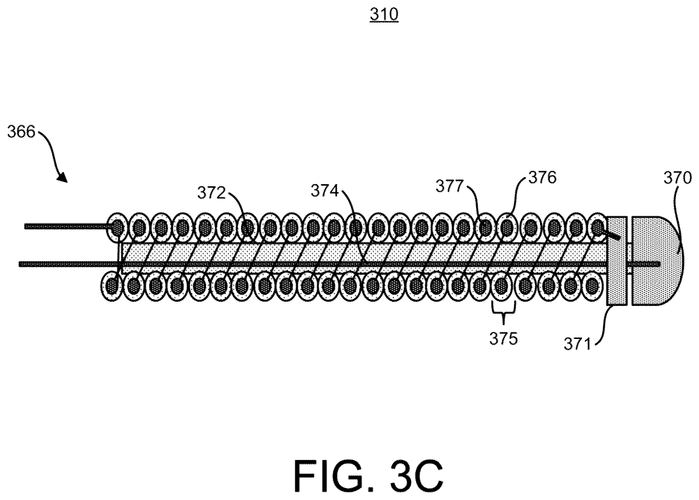

FIGS. 3A-3C are schematic illustrations of a distal end portion 366 of a RF perforation device 310 with a multi-layered insulated core providing dielectric potential to an energized guidewire, according to an embodiment of the present disclosure. The RF perforation device 310 may be substantially structurally and functionally identical to the RF perforation device 110 and 210 of FIGS. 1A-1C and FIG. 2, except as described in connection with FIGS. 3A-3C. As shown, the distal end portion 366 of the RF perforation device 310 terminates in a functional tip electrode 370. In embodiments, the tip electrode 370 may be localized at a desired target site, as explained above, and energy can be delivered from an energy source, e.g., an RF generator (not shown), through the RF perforation device 310 to the tip electrode 370 and the target site, thereby creating a void or perforation through the tissue at the target site.

In embodiments, as shown, the RF perforation device 310 further includes a conductor 374 that is insulated along a portion of its length and is electrically coupled to the tip electrode 370 and delivers RF energy to the tip electrode. In embodiments, the distal end portion 366 also includes a core 372 having a core lumen 378 extending longitudinally over the core 372. In embodiments, the functional tip 370 is coupled to the most distal end portion of the core 372. In embodiments, as shown in FIG. 3A, the core 372 includes the insulated conductor 374 extending longitudinally through the lumen 378 of the core 372. In embodiments, the core 372 may be made from using either a hypotube, metal coil wire, or a dielectric insulator with the insulated conductor 374 running longitudinally through the inner length of the core 372. Therefore, in other embodiments, the core 372 may not include the core lumen 378. In further embodiments, as shown, the distal end portion 366 may also include outer coil wires 375 that are positioned on the outside of the core 372. In embodiments, the outer coil wires 375 may run along the core 372 covering the wire, as shown in FIG. 3A. In other embodiments, the outer coil wires 375 may be positioned only at the most distal end portion of the core 372 near the functional tip 370.

In embodiments, the core 372 is nonconductive. In the illustrated embodiments, the core 372 is of a unitary construction formed entirely of an electrically insulative material. One exemplary class of electrically insulative materials for construction of the core 372 can include various grades of polytetrafluoroethylene (PTFE), fluorinated ethylene propylene (FEP), polyetheretherketone (PEEK), polyethylene terephthalate (PET), polyimide, among others. In embodiments, if the conductor 374 is itself insulated, the core 372 may be made from a hypotube, coil, braid, or polymer sleeve. An exemplary class of materials for the construction of the hypotube or metal coil wire of the core 372 may include various grades of stainless steel, nitinol, MP35N®, or Inconel. Any biocompatible electrically conductive material may be selected for construction of the conductor 374. Exemplary materials for construction of the conductor 374 may include copper, platinum iridium, copper cladded stainless steel, platinum core stainless steel, tungsten copper alloy, and molybdenum copper alloy, and the like. Furthermore, the conductor 374 within the lumen 378 is a thin insulated conductor. The hypotube or coil wires may be insulated from the core 372 and/or made from a non-conductive material.

Thus, in embodiments, the outer coil may include an outer insulative layer 376 and an inner conductive layer 377. Exemplary materials of the outer insulative layer 376 may include various grades of polytetrafluoroethylene (PTFE), fluorinated ethylene propylene (FEP), polyetheretherketone (PEEK), polyethylene terephthalate (PET), polyimide, among others. Exemplary materials of the inner conductive layer 377 may include copper, platinum iridium, copper cladded stainless steel, platinum core stainless steel, tungsten copper alloy, and molybdenum copper alloy, and the like.

In embodiments, the distal end portion 366 of the RF perforation device 310 may only include the conductor 374 delivering RF energy to the tip electrode 370 and the outer coil 375 providing insulation with its outer insulative layer 376 with no need for the core 372. Additionally, in embodiments, the core 372 may be made from a metal hypotube or coil wires with the coil 375 wound around the distal end portion of the wire near the functional tip 370 to provide radiopacity. Radiopacity is the state or degree of being opaque to X-rays or other radiation. In embodiments, the outer coil 375 may be made from a radiopaque metal covered with an insulated layer or made from a polymer doped with a radiopaque material. In embodiments, the outer coil wires 375 can be insulated to add another layer of protection from voltage leakage, but still have an inner metallic core to provide structural support and radiopacity.

In embodiments, as illustrated in FIG. 3B, the core 372 may be made from the metal core with the insulated conductor 374 running along a helix groove into the core 372 along its length. In embodiments, the core 372 has a helical groove manufactured into the core. In embodiments, if the conductor 374 is wound around the core 372 in the helical groove, the conductor 374 will relieve the tension created in the core when manipulated, due to the constant periodic nature of the pitch of the wire. In embodiments, the conductor 374 may have a smaller overall diameter than the conductor in FIG. 3A since the core 372 may provide the overall wire strength. In embodiments, the groove will allow another conductive wire to be fed to the distal end of the wire, which would enable the RF energy to be fed through the external insulated conductor 374 instead of feeding energy through the wire, as shown in FIG. 3A. In other embodiments, the core 372 may include overmolding with the conductor 374. In embodiments, the core 372 with the helical conductor 374 may further include outer coiled wires for further insulation and/or structural support and radiopacity.

In embodiments, the distal end portion 366 of the RF perforation device 310 may also include a second ring electrode 371, as illustrated in FIG. 3C, to create a bipolar structure. In embodiments, the conductor 374 may be electrically coupled to the tip electrode 370 and deliver RF energy to the tip electrode 370. As further shown, the distal end portion 366 may include a core 372 made of insulative material. In embodiments, the functional tip 370 is coupled to the distal end of the core 372. In further embodiments, the distal end portion 366 may also include outer coil wires 375 that are positioned on the outside of the core 372. In embodiments, the coil wires 375 may run along the core 372 covering the wire. In embodiments, the outer coils 375 may deliver RF energy to the ring electrode 371 for ablation or stimulation. In other embodiments, the outer coils 375 may be used for passive sensing.

Generally, in embodiments, the core 372, a structural member, is insulated from the conductor, 374, an inner conductive member, to provide good rail support with better dielectric insulative properties. In embodiments, the conductive material of the conductor 374 allows the RF energy to be applied to the functional tip electrode 370 for ablation. In embodiments, either the core 372 (with spiral groove) or the coil wires 375 would provide the rail support to deliver adjunct devices over the wire 374. In further embodiments, the smaller insulated conductor allows the RF energy to be fed through the smaller insulated wire instead of the entire larger core. This provides a safety feature as the entire core does not then need to be insulated with as thick of an insulative layer. In embodiments, this multiple layers of insulation on the inner core 372 and outer coiled wires 375 provides a high dielectric potential to the energized conductor 374 that may be used in any energized guidewire for ablation or stimulation. In other embodiments, the inner core 372 and the outer coiled wires 375 may be used for sensing.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A transseptal access system comprising:
a radiofrequency perforation device including a core, a functional tip coupled to a distal end of the core, and a conductor extending along a length of the core,
herein the conductor is insulated along a portion of its length and is electrically coupled to the functional tip.

2. The transseptal access system of claim 1, the core further comprising a core lumen.

3. The transseptal access system of claim 1, the radiofrequency perforation device further comprising an outer coil wound around a distal portion of the core.

4. The transseptal access system of claim 3, further comprising a second electrode, wherein the outer coil is electrically coupled to the second electrode.

5. The transseptal access system of claim 1, wherein the core is a hypotube.

6. The transseptal access system of claim 1, wherein the core is made from a non-conductive material.

7. The transseptal access system of claim 1, wherein the conductor is made from a conductive material and at least one of an outer surface of the core and an inner surface of the core is at least partially covered by an insulating material.

8. The transseptal access system of claim 3, wherein the outer coil is positioned on the distal most portion of the radiofrequency perforation device proximal to the functional tip.

9. The transseptal access system of claim 1, wherein the core includes a helical groove extending along an outer surface and further wherein the conductor is disposed at least partially in and along the helical groove.

10. The transseptal access system of claim 3, wherein the outer coil is made from a radiopaque metal material covered by an insulating layer.

11. The transseptal access system of claim 3, wherein the outer coil is made from a polymeric material doped with a radiopaque material.

12. The transseptal access system of claim 1, wherein the core is selected from the group consisting of polytetrafluoroethylene (PTFE), fluorinated ethylene propylene (FEP), polyetheretherketone (PEEK), polyethylene terephthalate (PET), and polyimide.

13. The transseptal access system of claim 1, wherein the conductor is selected from the group consisting of copper, platinum iridium, copper cladded stainless steel, platinum core stainless steel, tungsten copper alloy, and molybdenum copper alloy.

14. A transseptal access system comprising:
a radiofrequency perforation device including a core having a core lumen, a functional tip coupled to a distal end of the core, a conductor extending along a length of the core, and an outer coil wound around a distal portion of the core,
wherein the conductor is insulated along a portion of its length and is electrically coupled to the functional tip.

15. The transseptal access system of claim 14, further comprising a second electrode, wherein the outer coil is electrically coupled to the second electrode.

16. The transseptal access system of claim 14, wherein the core is a hypotube, and wherein the core is made from a non-conductive material.

17. The transseptal access system of claim 14, wherein the conductor is made from a conductive material and at least one of an outer surface of the core and an inner surface of the core is at least partially covered by an insulating material.

18. The transseptal access system of claim 14, wherein the outer coil is positioned on the distal most portion of the radiofrequency perforation device proximal to the functional tip.

19. The transseptal access system of claim 14, wherein the core includes a helical groove extending along an outer surface and further wherein the conductor is disposed at least partially in and along the helical groove.

20. The transseptal access system of claim 14, wherein the outer coil is made from a radiopaque metal material covered by an insulating layer, and wherein the outer coil is made from a polymeric material doped with a radiopaque material.
